(19) United States
(12) Patent Application Publication
(10) Pub. No.: US 2004/0219175 A1
(43) Pub. Date: Nov. 4, 2004

(54) Thermogelling emulsions for sustained release of bioactive substances

(75) Inventors: Pei Kan, Hsinchu (TW); Xi-Zhang Lin, Tainan (TW); Ming-Fa Hsieh, Hsinchu (TW); Ken-Yuan Chang, Hsinchu (TW)

Correspondence Address: BACON & THOMAS, PLLC, 625 SLATERS LANE, FOURTH FLOOR, ALEXANDRIA, VA, 22314

(73) Assignee: Industrial Technology Research Institute, Hsinchu (TW)

(21) Appl. No.: 10/748,192

(22) Filed: Dec. 31, 2003

(30) Foreign Application Priority Data
Dec. 31, 2002 (TW) 091138148

(51) Int. Cl. A61K 9/00

(52) U.S. Cl. 424/400

(57) ABSTRACT

The present invention relates to a temperature-sensitive thermogelling emulsion system for bioactive substances, consisting of a biodegradable temperature-sensitive aqueous phase polymer solution, and a pharmaceutically acceptable oil phase carrier, which embeds the bioactive substance. The oil phase carrier and the temperature-sensitive polymer solution are mixed mutually to produce the emulsion, and the emulsion is a liquid while the temperature is below the lower critical solution temperature (LCST) and reversibly transforms into a gel while the temperature is above said lower critical solution temperature. The emulsions are able to offer the controlled release without burst effect and afterward in a near zero order manner, as well as be non-invasively monitored post-implantation. The design of entrapment of the bioactive substances in the oily phase is also beneficial to preserve the bioactivity of the environment-sensitive substances.

### BACKGROUND OF THE INVENTION

1. Field of the Invention

The present invention relates to a delivery system for bioactive substances and, more particularly, to a bioactive substance delivery system using a temperature-sensitive emulsion.

2. Description of Related Art

The long-term sustained release system has the advantages of reducing injection frequency to enhance the patient's compliance, controlling steady drug release rate to eliminate the possible side effects and enhancing efficacy at specific local disease. When it was used for local therapy, the system is beneficial for drugs to accumulate in the target tissues or organs, at the same time, reducing the possible systemic toxicity caused by circulation of drugs throughout the body.

At present, several concepts have been developed to constitute a delivery system for long-term release of drugs, and the preferable method is the implantation of drug reservoir, or so-called depot, inside the body. In this aspect, the delivery system for said drugs requires a better biocompatibility to the surrounding tissue. Using biodegradable material is more convenient and comfortable for patients since an invasive surgical operation is not necessary to withdraw said remedy. Therefore, the biodegradable material is preferable and predominant for the long-term release administration system. However, the extremely acidic solution or organic solvent is often required for the preparation of the current biodegradable polymer systems. The condition is inappropriate for the macromolecules (e.g. proteins and peptides) processing and becomes a limit in the application of the delivery system for environment-sensitive macromolecules.

Recently, several polymers used for hydrogel applications have been developed. Since they contain a high percentage of water molecules, hydrogel can safely encapsulate the protein drugs without using extremely condition of aqueous solutions or organic solvents, and thus largely extending the applications. However, due to such a high water content in the hydrogel matrix, hydrophilic molecules diffuse out easily through the water-rich channel, most of the drugs are released rapidly in the early stage after the injection, which is the so-called ‘burst release’. For the drugs with strong potency, said burst effect could lead to drastic side effects among those treated subjects. Moreover, as most of the drugs released in the early stage, the duration time for afterward released drug level ranging within therapeutic window is shortened. In addition, one of the critical objects for the hydrogel system is to develop a system for long-term release of said drugs via a near zero-order release rate. Except for avoiding variation in release rate causing adverse effect, the steady release of drugs is also beneficial to extend the duration time when the drug level is within the therapeutic window.

Bioactive substances (e.g. drugs, growth factors, nucleic acids etc.) delivery is very important in the biomedical applications comprising tissue engineering, cell therapy and disease medical therapy. Those materials for the delivery carriers must provide biocompatible and biodegradable properties for the feasible implantation. Preferably, said material is a fluid ex vivo for easily mixing with drugs and transforms into a gel after being injected into the body by a syringe, catheter or laparoscope for delivering said bioactive substances to the desired tissue area. Currently, few delivery materials are satisfied to the requirement; some of them form gels via chemical reactions, which may influence the activity of bioactive substances and damage the implanted tissue. On the other hand, some of these materials possess temperature-sensitivity and gelling property but poor biodegradability, so they also fail to be implanted satisfactorily inside the body.

The biodegradable hydrogel used for drug release usually encapsulates drugs in the hydrogel matrix. The biggest problem of the system is that many bioactive substances, particularly for water-soluble drugs, peptides and proteins, fail to release in accompanying the polymer hydrolysis rate. In contrast, they diffuse out much faster through the water-rich channel in the hydrogel matrix. Therefore, the drug release rate bursts in the early stage, especially when using said hydrogel system for implanted drug reservoir treatment. The resulting burst release will cause side effects and be dangerous in the early stage of injection. Besides, the excessive drugs released in the early stage will cause insufficiency of the residual drug amount in depot required for the following long-term release.

In U.S. Pat. No. 6,287,588 Shih et al. developed a system for releasing bioactive substances in a controlled manner. The drugs were entrapped in the poly(lactic acid-co-glycolic acid) polymer (PLGA) microspheres, and then mixed with temperature-sensitive biodegradable hydrogel matrix (PLGA-PEG-PLGA triblock copolymer) to improve the drug release rate. Release rate was controlled by adjusting the composition and size of the microsphere. However, the major problem of the system is still involved in the organic solvent-related preparation for polymer microsphere. The organic solvents, necessary to be used during preparation, are harmful to most of drugs such as proteins, peptides, DNA and many solvent-fragile drugs. The bioactivity and potency will be damaged and lowered after the drug entrapment into the polymer microspheres. In addition, the hydrolysis of the PLGA polymer microspheres leads to acidic microenvironment inside and outside the microspheres. The pH value will drop to 1-2. The extremely acidic condition will damage the acid-fragile substances significantly. Therefore, the drugs may have lost the bioactivity as they released from the system even in a controlled manner.

### SUMMARY OF THE INVENTION

The object of the present invention is to provide a delivery system capable for delivering bioactive substances inside the body. Said delivery system comprises an oil phase to embed the therapeutic agents and then to mix with the aqueous phase of the temperature-sensitive hydrogel to form the thermogelling emulsion which is a fluid at room temperature and becomes a gel after temperature rise for the purpose of achieving long-term sustained release of the therapeutic agents.

To achieve the object, the present temperature-sensitive sustained release delivery system for bioactive substances comprises a biodegradable and/or bio-eliminating temperature-sensitive polymer, at least one bioactive substance, and a pharmaceutically acceptable oil phase carrier; said oil phase embeds said bioactive substances in a form of soluble in oil, solid-in-oil, water-in-oil or a mixture of them, and said oil phase further mixes mutually with said temperature-sensitive polymer solution to form an emulsion, which is a liquid while the temperature is below the lower critical solution temperature (LCST) and turns into a gel while the temperature is above said lower critical solution temperature (LCST). Said temperature-sensitive polymer provides the properties of being in a liquid state at low temperature and converting into a solid state with the temperature elevation as being injected into the body. For example, polymer PEG-PLGA-PEG is represented as formula (I):

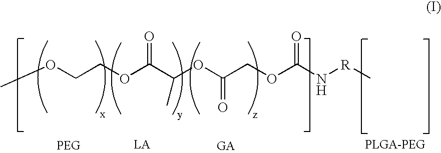

wherein x is a positive integer between 5 to 20; y is a positive integer between 20 to 40; z is a positive integer between 5 to 20; and R is the substituted linear or branched C₂ to C₁₀ alkyl group. In addition, diblock copolymer PEG-PLGA is represented as formula (II):

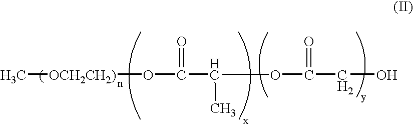

wherein n is a positive integer between 5 to 20; x is a positive integer between 20 to 40; and y is a positive integer between 5 to 20. Another triblock copolymer Poloxamer 407 is represented as formula (III):

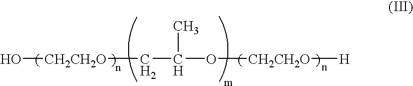

Materials with their liquid state in the lower temperature and in their solid state after the temperature rises are preferable in the present system. In addition, said oil phase carrier can be a fatty acid ester, preferably lipiodol, medium chain triglyceride (MCT), soybean oil, sesame oil, castor oil, sunflower oil, mineral oil, vitamin E oil or a mixture of them.

In the present invention, the bioactive substances (including small molecules, peptide or protein drugs) are embedded in the oil phase; said oil phase is mixed with the temperature-sensitive phase transition hydrogel solution to constitute a thermogelling emulsion system. Said system is in a liquid form at low temperature, preferably below 25° C., and becomes a gel form with the temperature elevation, preferably higher than 30° C. as in contact with the body. Said emulsion composition can alter the drug release profile after the gel formation. The formulation can prevent the burst effect and release the drug over a long-term duration in a steady release rate. In addition, the present delivery system can embed hydrophobic or hydrophilic bioactive substances, or a mixture of them in the meantime. Said drug release rate can be manipulated by the multi-compartments of emulsion entrapping the drugs, hydrogel hydrolysis rate and emulsion formulation. Environment-sensitive bioactive substances can be entrapped in the special compartment of the oily phase and prevented from acid condition caused by hydrolysis of hydrogel matrix. When phase contrast agent is used as inner aqueous phase in w/o emulsion or as the oily phase, the location and size of the present delivery system in the body can be non-invasively monitored post-implantation.

The present invention utilizes the synthetic temperature-sensitive biodegradable and/or bio-eliminating polymers. Said polymer solutions are mixed with the oil phase as thermogelling emulsions, and then injected into the body. The emulsions still preserve the temperature-sensitive property and transform reversibly between liquid and gel forms in response to temperature change. Said drugs are dissolved in oil, suspended in oil, or embedded in the water droplets in the oily phase to adjust the drug release profile to release the drug over a long term in a steady release rate as well as to prevent the burst effect in the early stage of injection. Additionally, this system also provides a protective effect on environment-sensitive bioactive substances and able to be non-invasively monitored post-implantation by X-ray examination.

Other objects, advantages, and novel features of the invention will become more apparent from the following detailed description when taken in conjunction with the accompanying drawings.

### BRIEF DESCRIPTION OF THE DRAWINGS

FIG. 1 is the NMR analysis data of the product of the embodiment 2 in the present invention.

FIG. 2 is the phase transition diagram of PEG-PLGA-PEG block copolymer versus time of the embodiment 3 in the present invention.

FIG. 3 is the phase transition diagram of PEG-PLGA block copolymer versus time of the embodiment 3 in the present invention.

FIG. 4 is the diagram of viscosity versus temperature for hydrogel formulation of the embodiment 4 in the present invention.

FIG. 5 is the diagram of viscosity versus temperature of thermogelling emulsions of the embodiment 4 in the present invention.

FIG. 6(A) shows the accumulated release amount within 33 days after administering the paclitaxel-containing hydrogel solution in the embodiment 5 of the present invention.

FIG. 6(B) shows the daily release amount within 33 days after administering the paclitaxel-containing hydrogel solution in the embodiment 5 of the present invention.

FIG. 7(A) shows the accumulated release amount within 33 days after administering the paclitaxel-containing thermogelling emulsions in the embodiment 6 of the present invention.

FIG. 7(B) shows the daily release amount within 33 days after administering the paclitaxel-containing emulsions in the embodiment 6 of the present invention.

FIG. 8 shows the accumulated release amount after administering the BSA-FITC-containing emulsions in the embodiment 14 of the present invention.

FIG. 9 shows the accumulated release amount after administering the doxorubicin-containing emulsions in the embodiment 15 of the present invention.

### DETAILED DESCRIPTION OF THE PREFERRED EMBODIMENT

For the greater understanding of the present art by those skilled in the art, there are eleven preferable embodiments specifically described as follows.

Embodiment 1 PEG-PLGA Polymerization

24.02 g of poly (ethylene glycol) (PEG, molecule weight is 550 g/mole), 50 g of lactide and 11.35 g of glycolide are added in the reactor, and the temperature is elevated slowly for complete dissolution. When the temperature reaches a continuous 110° C., 47.7 μl of catalyst (Stannous 2-ethyl-Hexanoate) is added and the reaction temperature is elevated and kept at 150° C. After polymerization is performed for 8 hr, the product is precipitated with diethyl ether/n-hexane (v/v=1/1) to form a translucent colloid. The residual monomers are washed for three times and dried in a vacuum for 24 hr at 40° C. The molecular configuration and molecule weight of the product are separately determined with NMR and GPC.

Embodiment 2 PEG-PLGA-PEG Polymerization

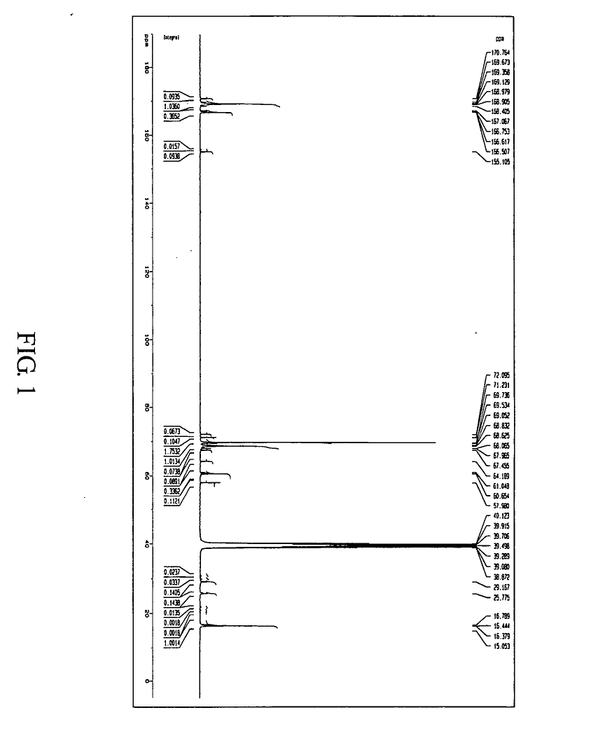

20 g of the product from embodiment 1 is put in a 250 ml round-bottom flask, and 200 ml of toluene is added into the flask followed by completely dissolving the mixture at 45° C. 1.73 ml (10.67 mmoles) of HMDI (Hexamethylene diisocyanate), and 0.71 μl of dibutyltin diacetate (initiator) is dissolved in 1 ml of toluene and then added into said flask after stirring evenly. After reaction for 12 hr at 60° C., the product is precipitated with diethyl ether/n-hexane (v/v=1/1). The residual is washed for three times and dried in a vacuum at 50° C. The molecular configuration and molecule weight are separately determined with NMR (as shown in FIG. 1) and GPC. The final polymer product is represented as formula (I).

Embodiment 3 Determination of Lower Critical Solution Temperature (LCST) of PEG-PLGA-PEG copolymer: Inverting Vial Method

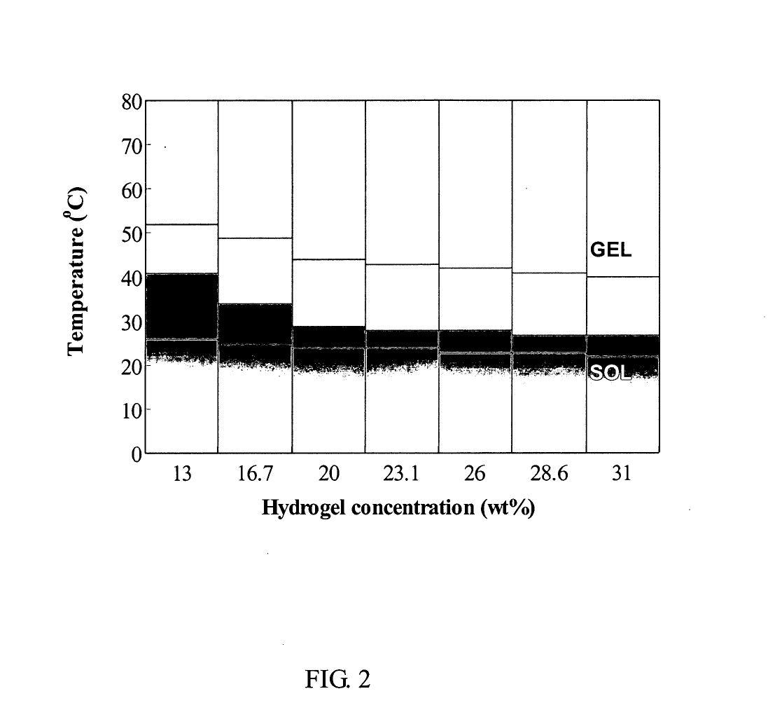

In the 4 ml transparent glass vials, the distilled water is used to prepare 15, 20, 25, 30, 35, 40, 45 w/v % of PEG-PLGA-PEG hydrogel and then stored at 4° C. refrigeration for the next step. A temperature controllable water bath is used to determine the lower critical solution temperature (LCST), and the initial temperature is 10° C. with an interval of 2° C. The glass vials are placed in a water bath for 5 min until the samples reach heat balance. Then the vials are taken out and stood upside down on a flat desk-top for 10-15 sec to observe their fluid patterns. From the above steps, it is called “sol” if the sample is still flowing and called “gel” if to the contrary. The result is illustrated in FIG. 2.

Embodiment 4 Determination of Lower Critical Solution Temperature (LCST) of PEG-PLGA copolymer: Inverting Vial Method

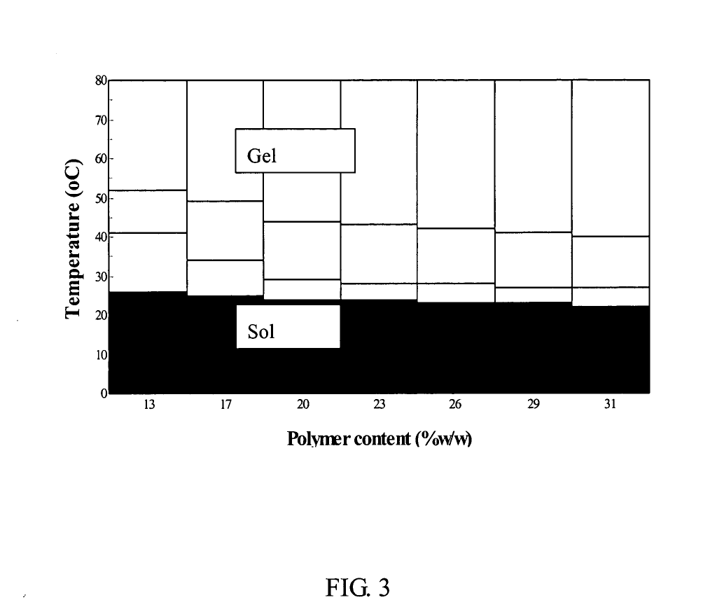

In the 4 ml transparent glass vials, the distilled water is used to prepare 15, 20, 25, 30, 35, 40, 45 w/v % of PEG-PLGA hydrogel and then stored at 4° C. for the next step. The determination was carried out following the same procedure described in embodiment 3. The result is illustrated in FIG. 3.

Embodiment 5 Determination of Time and Temperature Effect on PEG-PLGA-PEG Hydrogel

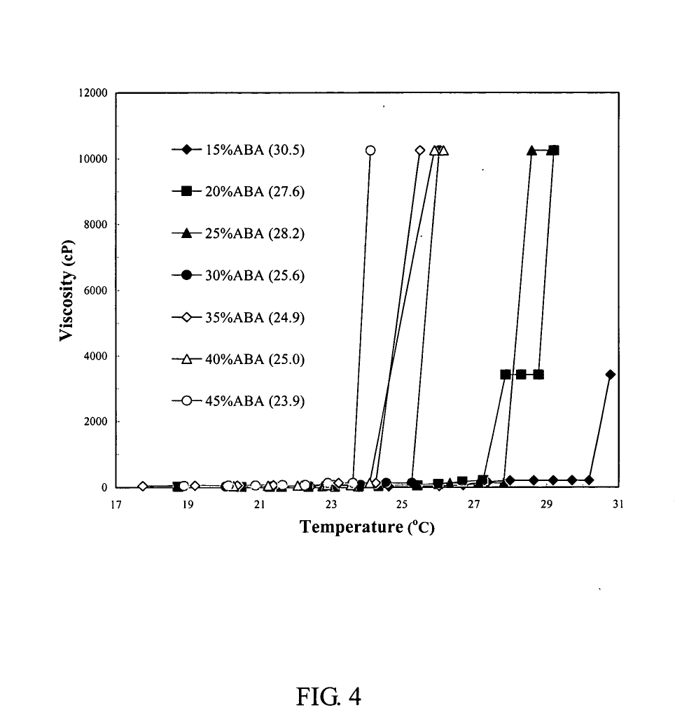

A Brookfield DVIII+cone and a plate Rheometer are used to determine the ex vivo gel formation time of the hydrogel. Before every test, the standard viscosity for said Rheometer is calibrated with standard solutions (100, 5000 and 10000 cP). 0.5 ml of said hydrogel is placed in the core of a plate with a temperature setting below 10° C. Said core bottom has a thermocouple to determine the sample temperature, and #CPD52 cone is used as a probe. In the beginning, 38° C. (or higher, but lower than 50° C.) of warm water is infused inside the plate, and the temperature of said plate arises dramatically to 36-38° C. Simultaneously, the special software for said Rheometer (Rheocalc) starts to record the data of hydrogel viscosity versus time, thermocouple temperature, rotational speed and torque of the Rheometer. In the determination step, said software adjusts the rotational speed automatically to maintain the torque between 80-100% for the authentic data. Said hydrogel gel formation time is the time required for the initial value of viscosity increasing to 10,000 cP in the experiment. The relationship between said single hydrogel agent and temperature is illustrated in FIG. 4.

Embodiment 6 Determination of Temperature Effect on PEG-PLGA-PEG Thermogelling Emulsions

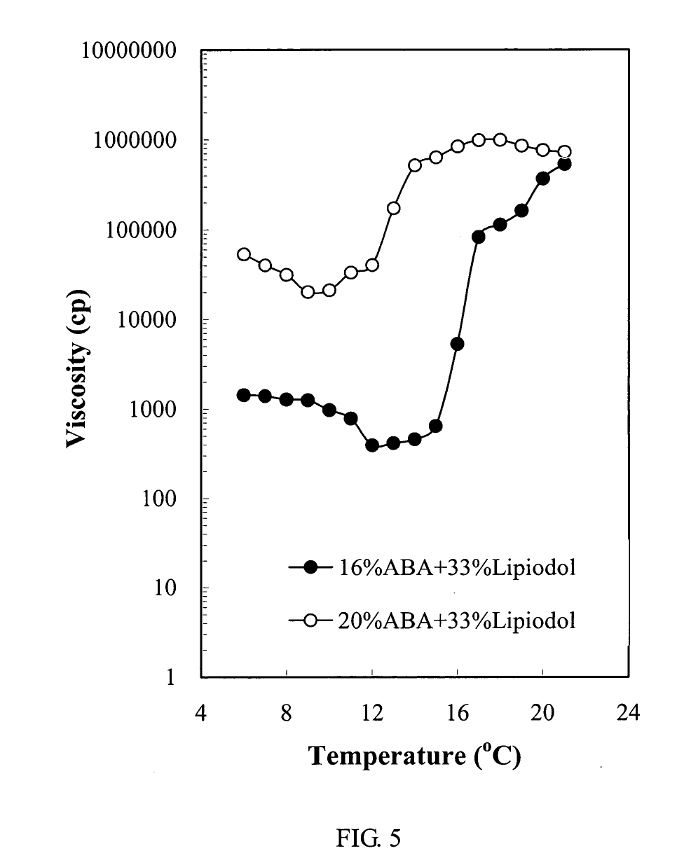

5 ml of 30% PEG-PLGA-PEG hydrogel and 4 ml of Lipiodol are mixed by vortex to form an emulsion, and then the variation of temperature to viscosity of said emulsion is determined by the Brookfield DVIII+cone and plate Rheometer. Before every test, the standard viscosity for said Rheometer is calibrated with standard solutions (100, 5000 and 10000 cP). 0.5 ml of said hydrogel is placed in the core of a plate with the temperature setting below 10° C., said core bottom has a thermocouple to determine the sample temperature, and #CPD52 cone is used as a probe. In the beginning, 38° C. (or higher, but lower than 50° C.) of warm water is infused inside the plate, and the temperature of said plate rises quickly to 36-38° C. Simultaneously, the special software for said Rheometer (Rheocalc) starts to record the data of hydrogel viscosity versus time, thermocouple temperature, rotational speed and torque of the Rheometer. In the determination step, said software adjusts the rotational speed automatically to maintain the torque between 80-100% for the authentic data. Said hydrogel gel formation time is the time required for the viscosity initial value to increase to 10,000 cP in the experiment. The relationship between said emulsion and temperature is illustrated in FIG. 5.

Embodiment 7 Determination of Temperature Effect on PEG-PLGA Thermogelling Emulsions

5 ml of 30% PEG-PLGA hydrogel and 2 ml of Lipiodol are mixed and shaken by a vortex to form an emulsion, and then stored at 4° C. for the next step. A temperature controllable water bath is used to determine the sol-gel inverting temperature, and the initial temperature is 10° C. with an interval of 2° C. The glass vials are placed in water bath for 5 min until the samples reach heat balance. Then the vials are taken out and stood upside down on a flat desk-top for 10-15 sec to observe their fluid patterns. From the above steps, it is called gel if the sample is not flowing. The result has shown that said emulsion represents gel formation patterns when the temperature is higher than 25° C.

Embodiment 8 Determination of Temperature Effect on Poloxamer 407 Thermogelling Emulsions

5 ml of 35% Poloxamer 407 hydrogel and 2 ml of Lipiodol are mixed and shaken by a vortex to form an emulsion, and then stored at 4° C. for the next step. A temperature controllable water bath is used to determine the sol-gel inverting temperature, and the initial temperature is 10° C. with an interval of 2° C. The glass vials are placed in water bath for 5 min until the samples reach heat balance. Then the vials are taken out and stood upside down on a flat desk-top for 10-15 sec to observe their fluid patterns. From the above steps, it is called gel if the sample is not flowing. The result has shown that said emulsion represents gel formation patterns when the temperature is higher than 30° C. It is indicated that different mixtures of temperature-sensitive polymers with oil phase can be used for said temperature-sensitive emulsion.

Embodiment 9 Release Study of Paclitaxel from the PEG-PLGA-PEG Hydrogels

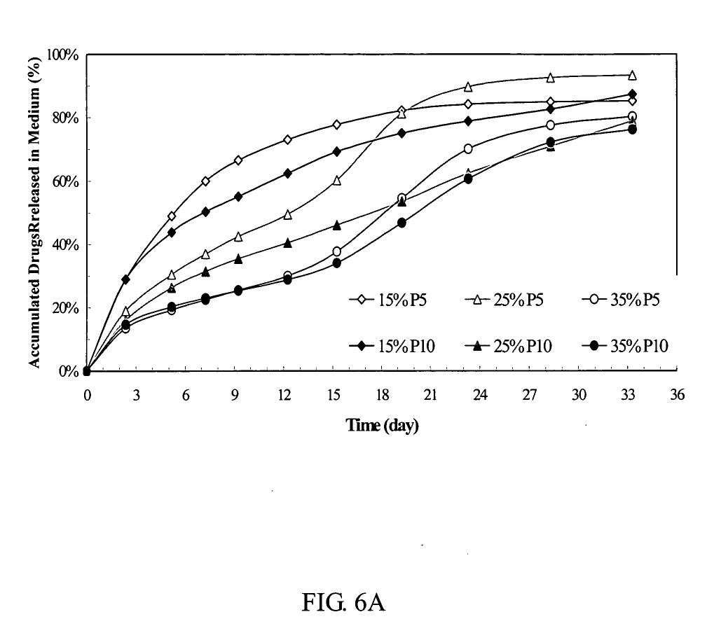

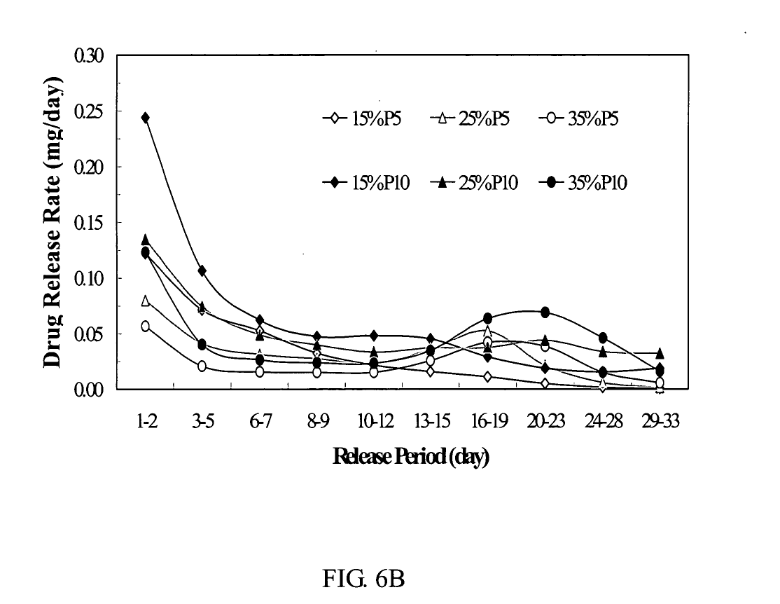

Under the low temperature, different components of PEG-PLGA-PEG are dissolved n water to form the hydrogel solutions and then a quantitative paclitaxel is added, and Vortex is used to shake and mix the mixture. Paclitaxel powders are suspended evenly in the solution under the low temperature. The hydrogel solution is maintained in a fluid status and 0.2 ml of said solution is added in a specific Release Cell by placing on the Thermstate Module and then stood for 10 min at 37.0±1.0° C. A sieve and a stirring bar (15 cm) are set, and 5 ml of the preheated Release Medium (37° C.) is added by a stirring speed of around 100 rpm. The Release Medium is renewed as the schedule and said samples are collected for analysis. The results are illustrated as FIGS. 6(A) and 6(B). FIG. 6(A) shows the accumulated release amount within 33 days from the hydrogel. It is indicated that 80% of the drugs are released by day 20 to day 30. The higher the polymer content is, the slower the release rate was obtained. The polymer content is one of the parameters for adjusting the release rate. In contrast, the amount of drugs did not influence the release rate. As shown in FIG. 6(B), a fast drug release at early stage was seen, and then followed with a stable drug release rate. A second release peak appeared since the fourth week started. 80% of paclitaxel in hydrogel were released in 3 weeks. Almost 90% of drug was released to the medium at the end of test period of 40 days. Concentration of polymer affected the release rate significantly but paclitaxel contents tested here did not.

Embodiment 10 Release of Paclitaxel from the Thermogelling Emulsion

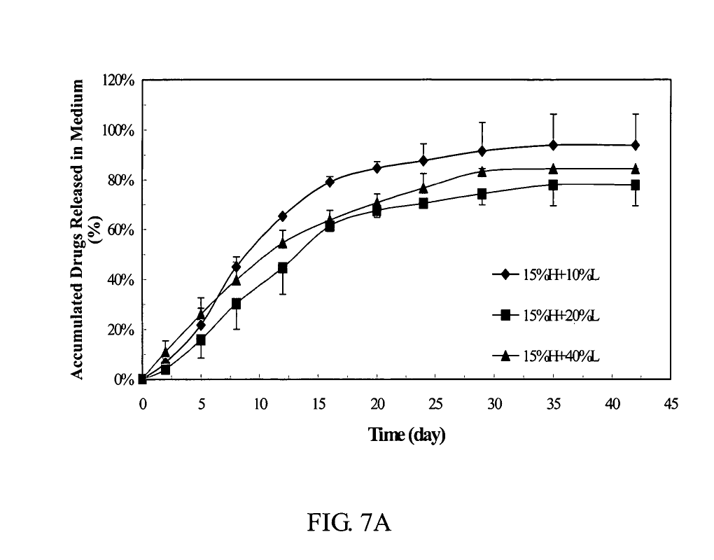

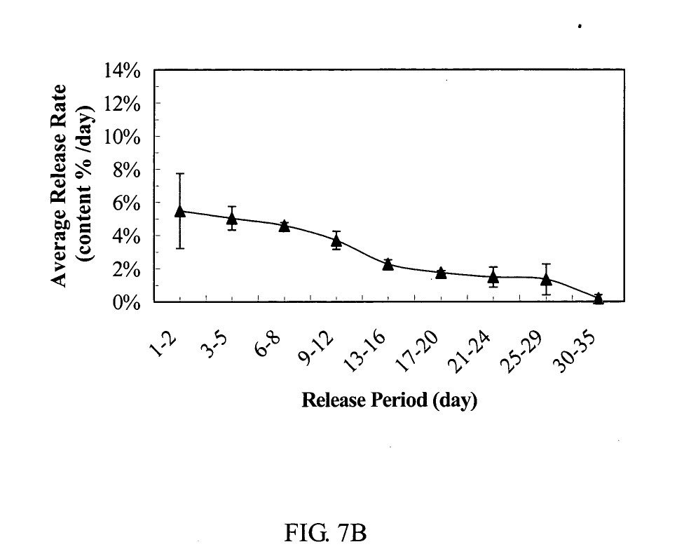

Under the low temperature, the quantitative paclitaxel is added in the lipiodol, and paclitaxel powders are suspended evenly in the oil phase. Different concentrations of PEG-PLGA-PEG hydrogel are mixed with the oil phase and then shaken by a Vortex to form the emulsion solution under low temperature. The emulsion is maintained in a fluid state and 0.2 ml of said solution is added in a specific Release Cell by placing on the Thermstate Module and then stood for 10 min at 37.0±1.0° C. A sieve and a stirring bar (15 cm) are set and 5 ml of the preheated Release Medium (37° C.) is added by a stirring speed of around 100 rpm to activate the release effect. The Release Medium is renewed as the schedule and said samples are collected for analysis. The results are illustrated as FIGS. 7(A) and 7(B). FIG. 7(A) shows the accumulated release amount within 33 days by hydrogel emulsion. It is indicated that 80% of the drugs are released by day 20 to day 30. FIG. 7(B) shows the daily release rate of the drugs illustrated by a percentage in the comparison with the initial amount. Initial burst effect was reduced as compared to the previous hydrogel system. Appropriate formulation of polymer and oil contents leveled off the variation of drug release and presented a near zero-order release rate. The release rate, therefore, was assumed to be controlled by drug diffusion from oily phase and polymer hydrolysis.

Embodiment 11 Preparation of Interferon-Containing Thermogelling Emulsions

Cremophore® RH-40, referred to as the surfactant able to stabilize w/o emulsions, was dissolved in the oily phase which could be median chain triglyceride, Lipiodol® or a mixture of them. Interferon was added in 1.8% NaCl, 0.3 mg/mL human serum albumin, 10 mM citrate buffer (pH 7) as aqueous phase. 2 volumes of aqueous phase were added drop by drop into 5 volume of oily phase, then being sonicated for 10 mins to form w/o emulsions. The mean particle size of the water drops in the oil was determined to be in the range from 120 to 500 nm by laser particle analyzer. The obtained w/o emulsion was mixed by hand with hydrogel solution to form interferon-containing thermogelling emulsions.

Embodiment 12 Preparation of w/o Emulsions for Thermogelling Emulsions

Cremophore® RH-40, referred to as the surfactant able to stabilize w/o emulsions, was dissolved in the oily phase which could be median chain triglyceride, Lipiodol® or a mixture of them. Bovine serum albumin conjugated with FITC (BSA-FITC), doxorubicin, or thymus DNA was dissolved in the buffered solution as aqueous phase, respectively. 2 volumes of aqueous phase were added drop by drop into 5 volume of oily phase, then being sonicated for 10 mins to form w/o emulsions. The water-soluble substances were then entrapped in the oil phase as the w/o emulsions. The mean particle size of the water drops in the oil was determined to be in the range from 120 to 500 nm by laser particle analyzer. The obtained w/o emulsion was mixed by hand with hydrogel solution to form w/o/w thermogelling emulsions.

Embodiment 13 Preparation of Protein Solids Suspended in Oil Phase for s/o/w Thermogelling Emulsions

An aqueous admixture of 5 mg bovine serum albumin and an amount (0-36 mg) of PEG 6000 was frozen onto a pre-cooled shelf of a freeze dryer at −50° C. Freeze-drying was performed by maintaining the shelf temperature at −20° C. for 3 hr and 20° C. for 12 hr under a pressure below about 50 mtorr. The obtained lyophilizate was dispersed in an appropriate solvent, which can dissolve PEG, but the proteins are insoluble. After centrifugation at 10,000 rpm for 5 min, the precipitates were collected and redispersed in the same solvent. This operation was repeated three times to remove the remaining PEG, and the final precipitates were dried under vacuum until they came to have a constant weight. The dry protein spheres were resuspended in the oil phase to constitute the protein-solid-in-oil solution. The oily phase was then mixed by hand with hydrogel solution to form s/o/w thermogelling emulsions.

Embodiment 14 Release Study of BSA-FITC from Thermogelling Emulsions

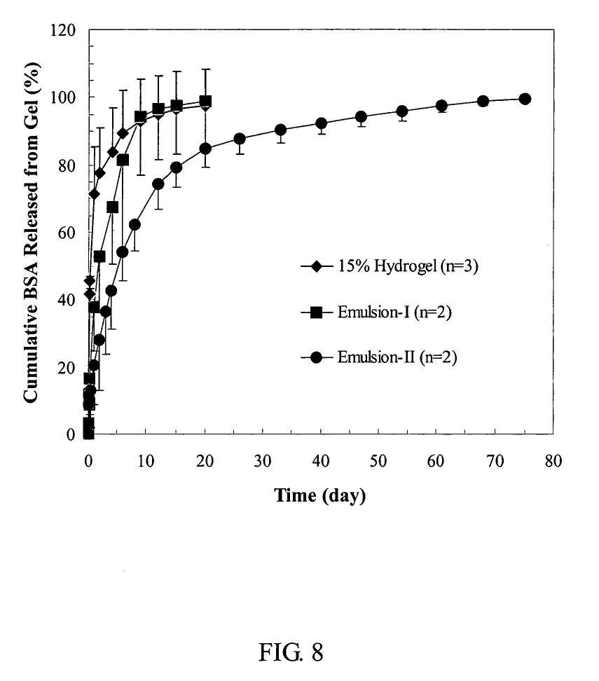

The BSA-FITC-containing thermogelling emulsions and hydrogels were maintained in a fluid status under low temperature before release test. 0.2 ml of said solution was added in a specific Release Cell by placing on the Thermstate Module and then stood for 10 min at 37.0+1.0° C. A sieve and a stirring bar (15 cm) are set and 5 ml of the preheated Release Medium (37° C.) is added by a stirring speed of around 100 rpm to activate the release effect. The Release Medium is renewed as the schedule and said samples are collected for analysis. The concentration of BSA-FITC released in medium was determined by fluorescent photometer. Formulation I used Span 80 as w/o emulsion stabilizer and formulation II cremophore® RH40, respectively As shown in FIG. 8, it took 2 days for hydrogel to release 80% BSA-FITC into the medium. Emulsions spent 6 and 20 days on reaching the same cumulative BSA-FITC for formulations I and II, respectively. The time of sustained-release version was extended by 3 to 10 folds. Burst release was also reduced by the emulsion formulation. 40% of BSA-FITC were released from hydrogel in the first 4 hours while less than 15% of BSA-FITC were released from emulsions within the same period.

Embodiment 15 Release of Doxorubincin from Thermogelling Emulsions

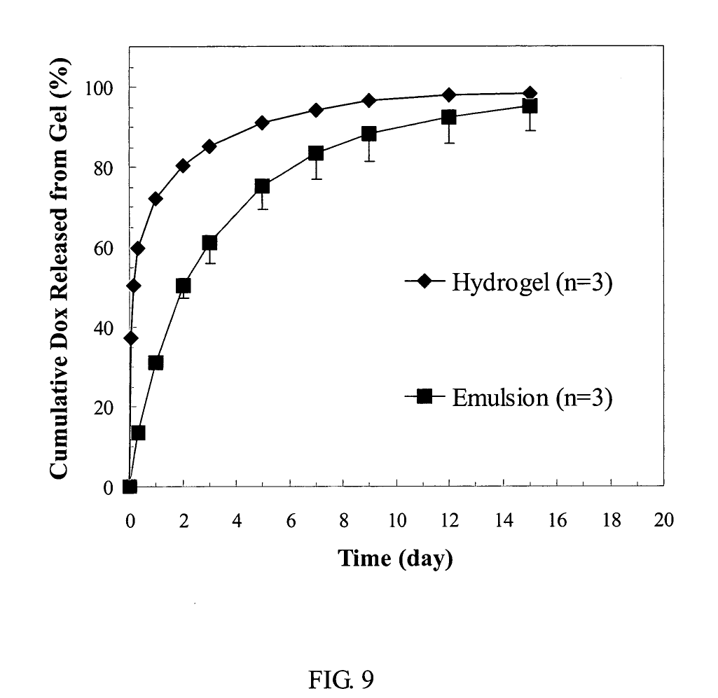

The doxorubicin-containing thermogelling emulsions and PEG-PLGA-PEG hydrogel were maintained in a fluid status under low temperature before release test. 0.2 ml of said solution was added in a specific Release Cell by placing on the Thermstate Module and then stood for 10 min at 37.0±1.0° C. A sieve and a stirring bar (15 cm) are set and 5 ml of the preheated Release Medium (37° C.) is added by a stirring speed of around 100 rpm to activate the release effect. The Release Medium is renewed as the schedule and said samples are collected for analysis. The concentration of doxorubicin released in medium was determined by fluorescent photometer. As shown in FIG. 9, it took 2 days for hydrogel to release 80% doxorubicin into the medium. Emulsions spent 6-7 days on reaching the same cumulative doxorubicin. The time of sustained-release version was extended by 3 folds. Burst release was also reduced by the emulsion formulation. 60% of doxorubicin were released from hydrogel in the first 8 hours while less than 14% of doxorubicin were released from emulsions within the same period.

Embodiment 16 Anti-Viral Effect of Interferon-Alpha-Thermogelling Emulsion on Hepatitis B Virus

The serum of the patient infected with hepatitis B virus was collected and injected into the SCID mice. Interferon-alpha, Interferon-alpha-containing emulsions and untreated groups of mice were divided to evaluate antiviral bioactivity of inteferon. Different formulations were administered at the interferon-alpha dose of 1000 units/g body weight of mice. Three days later, blood was collected and the concentrations of hepatitis B virus surface antigen (HBsAg) in plasma were determined by ELISA method. HBsAg in the untreated groups was defined as 1.0, the relative HBsAg ratio to the untreated control in the other groups were calculated and compared. The results indicated that the relative HBsAg in the thermogelling emulsion group was 0.26±0.7 and in the interferon-alpha group is 0.29±0.4, respectively. Interferon-containing thermogelling emulsion and interferon exhibited a statistically significant anti-viral effect (p<0.05) in compared to the untreated control. Also, thermogelling emulsion exhibited an undistinguishable anti-viral efficacy to interferon solution, indicating an appropriate preparation process for themogelling emulsion without observed damage on bioactivity of interferon.

Embodiment 17 Comparison of Bioactivity of Different Interferon-Alpha Formulations

After subcutaneous inoculation of human hepatoma cells (ATCC CRL-8024) to the SCID mice (1-2×10⁶ cells/mL), the tumors were about 0.5 cm in diameter in four weeks. The hepatoma cells are known to be infected by hepatitis B virus. Interferon-alpha-containing emulsions, hydrogel and untreated groups were divided to evaluate the efficacy of different interferon formulations. Different formulations were administered intratumorally at the interferon-alpha dose of 1000 units/g body weight of mice. One week later, blood was collected and the concentrations of hepatitis B virus surface antigen (HBsAg) in plasma were determined by ELISA method. HBsAg in the untreated groups was defined as 1.0; the relative HBsAg concentrations in the other groups were calculated and compared. The results indicated that the relative HBsAg in the hydrogel group was 0.96±0.04 and in the thermogelling emulsion group is 0.38±0.04, respectively. It demonstrated the protective effect of thermogelling emulsion on bioactivity of interferon-alpha is better than hydrogel only system.

Embodiment 18 Non-Invasive Observation of Thermogelling Emulsion in Animals

Lipiodol was added in the hydrogel solution and then shaken by a Vortex to form the thermogelling emulsion. Said emulsion was drawn into a syringe and injected subcutaneously in rats. Lipiodol was injected in another group of rats as the control., and the X-ray photograph analysis was performed one week post-injection. The result shown in FIG. 8 indicates that the rats injected with the thermogelling emulsion obviously indicate a depot under X-ray photography. In the case of group of rats injected with lipiodol only, said lipiodol rapidly flowed out and vanished, and therefore it could not be visible at the injection site on the following day by X-ray examination.

It is found from the above embodiments that the present bioactive substance delivery system will not only approach the temperature-sensitive gel formation, but also achieves long-term steady release of said carrying substance without burst release. Emulsion formulation also provides a protective effect on the bioactive substances entrapped in the hydrogel solution (such as PLGA-based copolymer) that creates acidic microenvironment. The location and size of said temperature-sensitive hydrogel inside the body can be detected in a non-invasive way post-implantation.

It is to be noted that in the present bioactive substance delivery system, the preparation method of said bioactive substance has no limit, and is preferably embedded in said oil phase carrier by the means of dissolving in oil, solid-in-oil suspension or water-in-oil emulsification. The present oil carrier can be a fatty acid ester, preferably Lipiodol, medium chain triglyceride (MCT), soybean oil, sesame oil, castor oil, sunflower oil, mineral oil, vitamin E oil or a mixture of them. The bioactive substance carried in the present invention is not limited, and is preferably at least one selected from the group consisting of chemical compound, virus, vector, protein, peptide, nucleic acid, polysaccharide, carbohydrate, lipid, glycoprotein and imaging agent. The way that the present bioactive substance delivery system enters into the body has no limit, and is preferably by subcutaneous, intramuscular, intraperitoneal, intracranial, intrathecal administration or vessel embolism agent.

Although the present invention has been explained in relation to its preferred embodiment, it is to be understood that many other possible modifications and variations can be made without departing from the spirit and scope of the invention as hereinafter claimed.

What is claimed is:

1. A temperature-sensitive thermogelling emulsion delivery system, comprising:
a biodegradable temperature-sensitive aqueous phase polymer solution;
at least one bioactive substance, and
a pharmaceutically acceptable oil phase carrier, said oil carrier embeds said bioactive substance;
wherein
said oil phase carrier and said temperature-sensitive polymer solution are mixed mutually to form an emulsion, which is a liquid while at a temperature below a lower critical solution temperature (LCST) and transforms into a gel while the temperature of the emulsion is above said lower critical solution temperature (LCST).

2. The delivery system as claimed in claim 1, wherein said bioactive substance is embedded in said oil phase carrier by the means of dissolving, solid suspension or water/oil emulsification.

3. The delivery system as claimed in claim 1, wherein said temperature-sensitive polymer is selected from the group consisting of PEG-PLGA-PEG, PLGA-PEG-PLGA, PEG-PLGA and Poloxamor 407.

4. The delivery system as claimed in claim 3, wherein said PEG-PLGA-PEG is represented as formula (I): 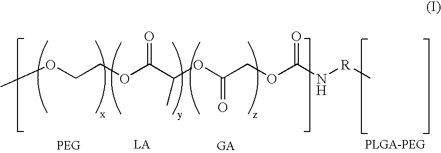
wherein x is a positive integer between 5 to 20; y is a positive integer between 20 to 40; z is a positive integer between 5 to 20; and R is the substituted linear or branched C₂ to C₁₀ alkyl group.

5. The delivery system as claimed in claim 3, wherein said PEG-PLGA is represented as formula (II): 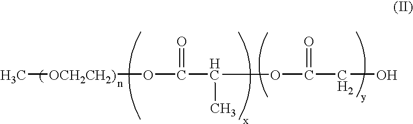
wherein n is a positive integer between 5 to 20; x is a positive integer between 20 to 40; and y is a positive integer between 5 to 20.

6. The delivery system as claimed in claim 3, wherein said Poloxamer 407 is represented below:

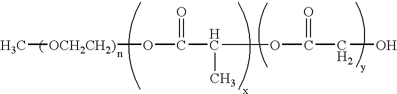

7. The delivery system as claimed in claim 1, wherein said physiologically accepted oil phase carrier is a fatty acid ester.

8. The delivery system as claimed in claim 7, wherein said physiologically accepted oil phase carrier is selected from the group consisting of lipiodol, medium chain triglyceride (MCT), soybean oil, sesame oil, castor oil, sunflower oil, mineral oil, vitamin E oil or a mixtire of them.

9. The delivery system as claimed in claim 1, wherein at least one bioactive substance is selected from the group consisting of chemical compound, protein, peptide, nucleic acid, polysaccharide, carbohydrate, lipid, glycoprotein and imaging agent.

10. The delivery system as claimed in claim 1, which is used for subcutaneous injection, intramuscular injection, intratumor injection or embolism agent.
